(19) United States
(12) Patent Application Publication
(10) Pub. No.: US 2018/0146908 A1
(43) Pub. Date: May 31, 2018

(54) DEVICE AND METHOD FOR MEASURING THE PERCEPTION OF EQUILIBRIUM IN INDIVIDUALS

(71) Applicant: CHRONOS VISION GMBH, Berlin (DE)

(72) Inventor: Friedrich-J. BAARTZ, Berlin (DE)

(21) Appl. No.: 15/545,465

(22) PCT Filed: Jan. 25, 2016
(86) PCT No.: PCT/DE2016/000026

§ 371 Date: Dec. 5, 2017

(30) Foreign Application Priority Data
Jan. 27, 2015 (DE) 10 2015 101 110.7

(51) Int. Cl. A61B 5/00; A61B 5/11; A61B 5/05

(52) U.S. Cl. CPC A61B 5/4023; A61B 5/11; A61B 5/05; A61B 5/1126; A61B 5/7445; A61B 5/1116

(57) ABSTRACT

The invention relates to a device for measuring the perception of equilibrium in individuals comprising an opaque mask having an inclination sensor and a display arranged within the mask for displaying an image, which can be rotated in order to determine the subjective visual vertical of an individual, and having a means for detecting the posture of the individual, and a monitoring unit which, during the determination of the subjective visual vertical, indicates and/or stores the position and/or movement of one or more areas of the body. During the measurement of the sense of equilibrium, the subjective visual vertical is determined and, at the same time, the posture of the individual is measured.

The invention relates to a device for measuring equilibrioception in accordance with the preamble of patent claim 1, to a method for measuring equilibrioception in accordance with the preamble of patent claim 9, and to the use of a device in accordance with patent claim 18.

The otolith organs in the equilibrium organ of humans contribute significantly to the correct perception of the body's orientation in space. They are made up of the utricle and the saccule. A disturbance of the otolith organ function can result in misjudgments relating to the body's orientation, to dizziness and to an unsteady gait with a tendency to fall.

The otolith organs, or otoliths in short, are located in the head and operate in accordance with the principle of an inertia sensor. In the case of linear acceleration, the otoconia, which are connected to the sensory hairs and are formed from crystals, are slightly displaced and trigger nerve excitations in the sensory cells. In normal, everyday situations, it is frequently Earth's gravity that acts on the otoliths when the head is tilted and thus triggers nerve excitation by way of the sensory cells. In other words, everyday head tilts relative to the space vertical are detected by way of the otolith organs. This provides an important piece of information for coordinating the body and for upright posture of the body.

For example, if a person in the dark, i.e. without visual orientation means, is asked to adjust a visible light-emitting strip or light-emitting line such that it is vertical or perpendicular in space according to that person's perception, highly reproducible results are obtained that, in healthy humans, largely correspond to the actual vertical in space, i.e. to the direction of gravitational force. The result from this measurement is referred to as “subjective visual vertical” (SW) and is made possible by way of the information from the otolith organs. The SVV thus characterizes the vertical orientation, or the up-down orientation, in space as subjectively perceived by a person. It is thus possible to use the results that are provided by the measurement of the SW for subsequent clinical examination of the otolith function.

The publication “Unilateral examination of utricle and saccule function,” by A. H. Clarke et al., Journal of Vestibular Research 13 (2003) 215-225, describes a measurement system and a method for determining the subjective visual vertical, in which a person looks into a dome in which a light line appears while the person rotates together with the dome about the body axis. The person then rotates the light line until he or she perceives it as being vertically aligned.

The European Patent application EP 0 363 521 A1 describes an apparatus for examining the function of the otoliths, comprising a darkened pair of spectacles having a tilt sensor, which are able to be placed on the head of a person. Located in front of the eye of the person, on the spectacle frame, is a measurement insert by way of which a first light line, which generates a line-type afterimage on the subject's retina, is provided via a gap using high-intensity radiation or a flash. When the head is tilted, a second light line is generated using low-intensity radiation via the same gap. The person now uses an adjustment flap at the spectacles to rotate the gap and thus the second light line, which is generated by way of the low-intensity radiation, until said second light line coincides with the afterimage of the first light line on the retina.

Specification DE 102012001981A1 describes a device for examining the otolith function and a method for determining the subjective visual vertical. The known device is shown in FIG. 2. The device comprises an opaque mask (10) which is able to be placed on a person in a manner such that it is light-proof, and a tilt sensor (18), the signals of which represent the tilt of the mask and/or any acceleration acting on the mask. An electronic display (12) serves for presenting an image inside the mask (10), wherein during operation, a vertically aligned image orientation is visually adjusted on the display (12) by way of control signals, in order to determine the subjectively perceived visual vertical, an image is presented on the display (12) within the opaque mask (10), wherein no light enters the interior space (9) of the mask (10) from the outside. The tilt of the head or of the mask is measured. The image on the display (12) is rotated by way of control signals about the observation direction B, B′ until it appears to the person to be vertically aligned in space.

Currently, disturbances of the sense of balance have been associated with disorders of the equilibrium organ. The otolith function in the equilibrium organ is checked by determining the subjective visual vertical, with the measurement result serving as a basis for diagnosing a disorder of the equilibrium organ.

The problem here is that other factors outside the equilibrium organ are not taken into consideration, or are insufficiently taken into consideration. Under some circumstances, this can result in falsification or uncertainties and misinterpretation in the later diagnosis.

The object of the invention is to solve this problem and to improve the informative value of the measurement of equilibrioception while reducing or avoiding uncertainties in the later diagnosis.

The object is achieved by way of the device for measuring equilibrioception in accordance with patent claim 1, by way of the method for measuring equilibrioception in accordance with patent claim 9, and by way of the use of a device in accordance with patent claim 18. Further advantageous features and details can be gathered from the dependent patent claims, from the description, and from the drawings.

The device according to the invention comprises an opaque mask having a tilt sensor or inertial sensor and a display, arranged inside the mask, for presenting an image that can be rotated in order to determine the subjective visual vertical of a person, and an apparatus for capturing the body posture and/or body movement of the person, and a monitoring unit that displays and/or saves the orientation and/or movement of one or more body regions during the determination of the subjective visual vertical.

By rotating the image, its vertical or horizontal image orientation is adjustable.

It is also possible using this device to diagnose, during the measurement of the subjective visual vertical (SVV), due to the simultaneous capturing of the orientation of body parts, illnesses that influence the SVV perception, without a disorder of the equilibrium organ being present. In particular, orthopedic disorders can be diagnosed that have an impact on the sense of balance.

The apparatus for capturing the body posture advantageously comprises one or more sensors, which measure the alignment and/or movement of the body or of a plurality of body parts.

The apparatus for capturing the body posture can comprise one or more cameras that capture the alignment and/or movement of the body or of one or more body parts, and in particular capture markers provided on the body.

The at least one sensor is preferably in the form of an inertial sensor or an acceleration or angular rate sensor, which can also be combined, for example, with a magnetic field sensor.

By using one or more magnetic field sensors, the accuracy of the measurement is increased. For example, the alignment of the body parts or body regions which are provided therewith in the Earth's magnetic field can be ascertained in the process. This allows calculation in particular of the absolute orientation in space. They supplement, for example, acceleration sensors that determine the orientation of the body parts with respect to the gravitational acceleration vector or up to one rotation about the gravitational acceleration vector.

The body posture captured by the apparatus, or a characteristic parameter thereof, is preferably graphically displayed. The display is shown in particular on the display inside the mask. The person thus obtains the possibility of feedback in the form of the graphic display of the body posture on the internal display of the mask or spectacles, such as for example the two-dimensional distance of the body's center of gravity from the body axis.

The display of the captured body posture or of a parameter that is characteristic thereof can also be shown on a display outside the mask. This offers the possibility of providing feedback to the operator or treatment staff relating to the current position of the subject or patient, or also an indication as to what the subject or patient still needs to change in order to be able to assume the desired measurement position.

The monitoring unit is preferably configured to determine, from the signals of the sensors during the measurement, the position or orientation of individual body regions relative to one another, in particular the relative orientation between head and trunk. It is thus possible to perform a measurement of the SVV at an exactly defined or determined angle for example between head and trunk.

This gives in particular new diagnostic options. In SVV measurements until now, care has been taken that head and trunk are located in one axis. The device according to the invention can in particular be used to determine a value of the SW that deviates from the normal value, for example if the cervical spine is bent.

In particular, the monitoring unit is configured such that it determines, from the signals of the magnetic field sensors, the angles of one or more body regions with respect to a magnetic field, and ascertains therefrom the orientation of the body regions relative to one another. For example, the Earth's magnetic field can be used as the magnetic field. The use of magnetic field sensors or compasses permits, in combination with inertial sensors, the ascertainment of the absolute orientation of the sensors and thus of the associated body parts in space.

The device preferably comprises one or more of the following features:
(a) the opaque mask is configured to be placed on a person such that it is light-proof;
(b) the signals of the inertial sensor or tilt sensor represent the tilt of the mask with respect to the gravitational direction and/or an acceleration that acts on the mask;
(c) the vertically or horizontally aligned image orientation on the display is visually adjustable during operation by way of control signals, wherein a portable operating unit generates the control signals;
(d) provided is an evaluation unit that compares, in the case of an image orientation that appears to be aligned vertically, horizontally, or at a different predefined angle, the tilt angle of the image relative to the mask to the signal of the tilt sensor;
(e) the tilt sensor is integrated in the mask and is in the form of a three-dimensional acceleration sensor;
(f) the image is in the form of a light pattern, a light line, an image sequence, or a video;
(g) an image memory is integrated in the mask.

In the method according to the invention for measuring the equilibrioception of persons, an image is provided inside an opaque mask to a person, with the orientation of the image being adjusted such that it appears to the person to be aligned vertically in space, wherein a tilt sensor is used to capture the tilt of the head and/or an acceleration acting on the head in order to determine the subjective visual vertical of the person, wherein the body posture of the person is captured and the orientation and/or movement of one or more body regions of the person is displayed and/or saved by way of a monitoring unit.

The advantages and features mentioned with respect to the device also apply to the method according to the invention, and vice versa.

The orientation of the subjective visual vertical is preferably ascertained in dependence on the body posture.

The determination of the subjective visual vertical preferably takes place during a defined body posture which is displayed or checked by way of the monitoring unit.

In the case of a first body posture and in the case of a second body posture, advantageously, the respective orientation of the subjective visual vertical is measured, and both results are compared to one another.

During the measurement, the orientation of individual body regions relative to one another is preferably determined, wherein in particular the relative orientation of head and trunk is determined.

The captured body posture, or a characteristic parameter thereof, is advantageously graphically displayed, wherein the display is shown on the display inside the mask and/or on a further display outside the mask.

The image is in particular presented on an electronic display inside the mask, and the image is rotated, using control signals, about the observation direction B, B′, until it appears to the person to be aligned vertically in space.

The person preferably adjusts the subjectively perceived vertical image orientation him- or herself using a portable operating unit.

A device according to the invention can in particular be used during the performance of the method according to the invention.

According to the invention, the device according to the invention for measuring the effect of the body posture on the sense of balance is used in order to be able to ascertain disorders outside the equilibrium organ.

The term “vertically aligned” or “vertically aligned image orientation” is to be understood to mean that the person wearing the mask subjectively perceives the image orientation or the orientation of an imaged object in space as being vertically aligned merely by observing the image and without other reference points. That means that “up” and “down” in the image also exactly correspond to “up” and “down” in space for the person in this case. The vertically aligned image orientation is here also considered to be equivalent to a horizontal or otherwise previously determined alignment, for example in the case of an image object or image which extends horizontally or at any desired, previously determined angle, and is intended to comprise it in its definition. Subjective vertical alignment generally means that a vertical or a horizontal or a line extending at a previously determined angle in the image is perceived by the observer exactly as being aligned vertically or horizontally or at the otherwise previously determined angle in space.

The invention will be described below by way of example with reference to the drawings, in which:

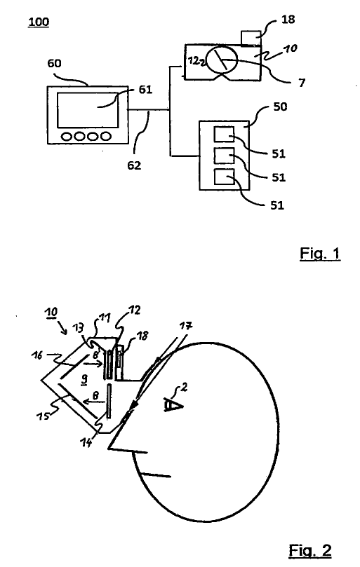

FIG. 1 shows a schematic illustration of a device according to a first preferred embodiment of the invention;

FIG. 2 shows a known mask for determining the subjective visual vertical, which mask is used as part of the device according to the invention;

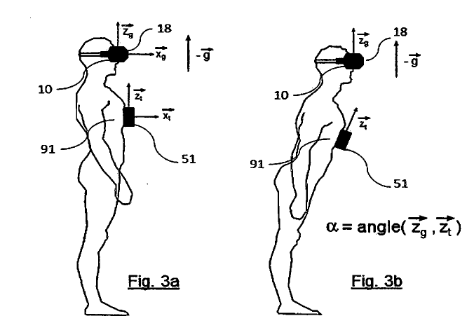

FIGS. 3a and 3b show a schematic illustration of a device according to a first preferred embodiment of the invention which is attached to a person having an upright body posture and a person having a tilted body posture;

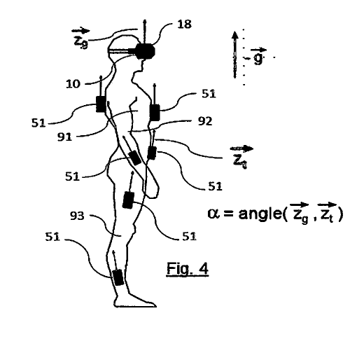

FIG. 4 shows a device according to a second preferred embodiment of the invention, which is arranged on a person, having a multiplicity of sensors;

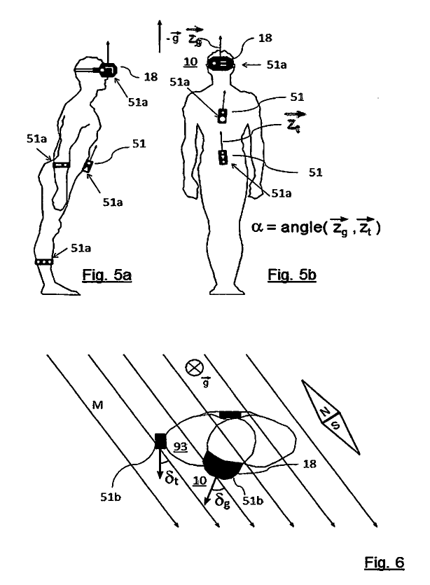

FIGS. 5a and 5b show a third preferred embodiment of the invention, wherein cameras are used;

FIG. 6 shows a schematic view from above of a third preferred embodiment of the invention, wherein magnetic field sensors are used;

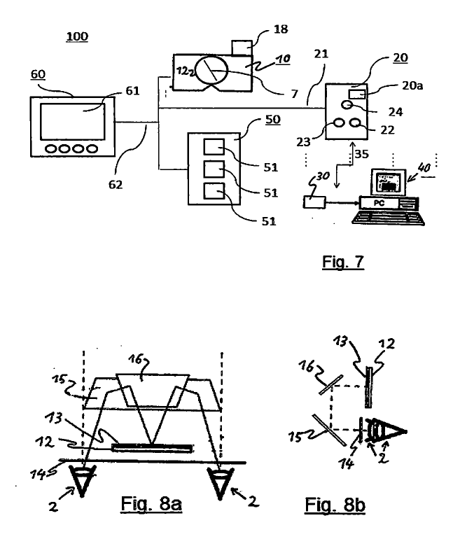

FIG. 7 shows a schematic illustration of the device according to the invention as a measurement system;

FIGS. 8a and 8b schematically show the beam path in the mask of the device according to the invention in a view from above or from the side; and

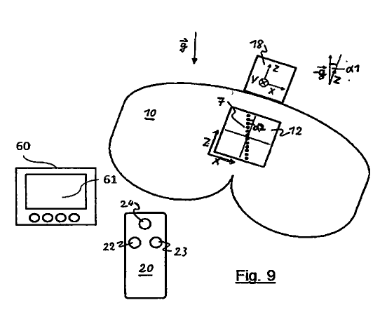

FIG. 9 schematically shows the view of a mask from the inside, having a monitoring unit and a handheld unit, for explaining the method according to the invention.

FIG. 1 shows a schematic illustration of a device 100 for checking the equilibrioception according to a first preferred embodiment of the invention, The device 100 comprises an opaque mask 10 for determining the subjective visual vertical, which mask is able to be placed on a person such that it is light-proof. An apparatus 50 for capturing the body posture of the person comprises one or more sensors 51, which are in the form of inertial sensors or of inertial sensors in combination with magnetic field sensors, and are attachable to body parts of the person. A monitoring unit 60 is electrically or wirelessly coupled, by way of a connection 62, to the apparatus 50 for capturing the body posture of the person.

The monitoring unit 60 comprises a display 61 and serves for displaying or saving the orientation of one or more body regions, while the subjective visual vertical, or SVV for short, is determined. A display 12 is arranged in the mask 10 for determining the SVV. Provided on the display 12 in the mask 10 is, during the measurement, an image 7 which by way of rotation is brought into a position that appears to the person to be vertically aligned. The mask 10 furthermore comprises a tilt sensor 18, which is for example an acceleration sensor or an inertial sensor and measures the tilt of the mask or an acceleration acting on the mask.

FIG. 2 shows in detail the mask 10 that is known from specification DE 10 2012 001 981 A1 and can be used as a component part of the device 100 according to the invention. The mask 10 comprises an opaque housing 11 and is placed on a person during operation for the purpose of measurement such that it is located in the region of the face in front of the eyes 2 of the person. In the worn state, the mask 10 is rigidly connected to the head such that it is light-proof, i.e. no light from the outside can enter the interior 9 of the mask 10 and thus the eye 2.

The tilt sensor 18 is integrated in the mask 10. The tilt sensor 18 is attached to the light-proof housing 11 and is in the form of a three-dimensional acceleration sensor. It provides signals that represent the respective tilt of the head or generally an acceleration acting on the head. Furthermore located and integrated in the mask 10 is the electronic display 12, which serves for presenting the image 7 (see FIG. 1) inside the mask 10 and is visible to the person wearing the mask 10. The display 12 is controlled by corresponding control signals such that the provided image 7 rotates about the observation direction B, B′. In this way, the image 7 is aligned vertically for the observer, that is to say during the measurement, the image orientation is adjusted by way of rotation about the observation direction such that the observer subjectively perceives it to be aligned vertically in space.

The mask 10 is embodied in the form of a pair of spectacles. In order to ensure the light-proof nature of the spectacles or of the mask 10 in the worn state, an elastic element 17 is provided as a face connection. The elastic element 17 is formed, for example, from a dark, opaque foam material, rubber or the like. It is located at the rim of the mask 10, which forms the contact with the face surface, and can be configured such that it is removable.

Arranged in the interior 9 of the mask 10 is a mirror device consisting of a main mirror 15 and a display mirror 16. The two mirrors 15 and 16, which are in the form of surface mirrors, are arranged such that the image or the light pattern provided on the display 12 is directed toward the eye 2 of the observer or of the person to be examined. The display 12 is here arranged in the beam path on the side of the opening in the housing 11 through which the person looks into the interior 9 of the mask 10. In contrast, the two mirrors 15, 16 are arranged on the opposite side, such that they reflect the image on the display 12 back to the eye 2 of the observer, wherein the beam path from the display 12 to the mirror device 15, 16 extends parallel to the beam path between the mirror device 15, 16 and the eye 2 of the observer.

Arranged in front of the display 12 is a diffuser element 13, which is preferably configured to be flat or plate-shaped. The diffuser element 13 prevents the observer from orienting him- or herself by way of image pixels which become visible as step-type patterns, for example, during the presentation of lines that extend at an angle in the image. The diffuser element 13 rules out the observer being able to draw conclusions with respect to the actual vertical orientation of an image element in space.

Located between the observation opening in the housing 11 and the mirror device is a Fresnel lens 14 for focusing the image.

FIGS. 3a and 3b show a schematic illustration of the mask 10 and the apparatus 50 for capturing the body posture in accordance with a first preferred embodiment of the invention in a state in which they are attached to a person having an upright body posture (FIG. 3a) and having a tilted body posture (FIG. 3b). Integrated as the tilt sensor 18 (see FIG. 1) in the mask 10 is an inertial sensor or acceleration sensor. A further sensor 51, which is likewise in the form of an inertial or acceleration sensor, is arranged at the torso 91 of the person.

The sensors 18 and 51 measure the angles between their respective axes and the gravitational vector g or gravitational acceleration. From this, it is possible to calculate the angle between the Z axes or vertical axes (Z_{g}, Z₁) of the two sensors 18 and 51 if the person is looking straight ahead. By ascertaining the angle between the Z axes of the sensors 18 and 51 and the gravitational vector g, it is possible for example to deduce that the torso 91 or trunk is leaning forward and to capture this in the form of a measurement variable.

FIG. 4 shows a device according to a second preferred embodiment of the invention, having a multiplicity of sensors 51, which are illustrated by way of example as being arranged on a person and which capture the body posture or the orientation or alignment of different body regions. Illustrated as arrows are the Z axes of the respective sensors 51 which, in this example, are attached to the front and back of the torso 91 and to the arms 92 and legs 93 of the person and measure the orientation thereof relative to the gravitational vector g.

FIGS. 5a and 5b show a third preferred embodiment of the invention, in which cameras are used to capture the body posture. For this purpose, markers 51a, which are located on the body of the person and on the mask 10, are attached in place of sensors 51 or in addition to the sensors 51. By way of one or more cameras (not illustrated in the figures) and image processing, the orientation and alignment and, if required, also the movement of the markers on the body and on the mask 10 are ascertained.

FIG. 6 shows a schematic view from above of a third preferred embodiment of the invention, wherein magnetic field sensors 51b are additionally arranged, which are located in the direct vicinity of the sensors 51 or are integrated therein. In this example, the magnetic field sensors 51b are attached to the shoulder 93 of the person and to the mask 10, wherein they are integrated in the previously described sensors 51. The field lines of the Earth's magnetic field M are illustrated in the form of arrows.

By positioning the magnetic field sensors 51b, the determination of the angles δ_{g}, δ₁ between the respective axes of the sensors and the field lines of the Earth's magnetic field M is made possible. From this it is possible to ascertain, in combination with inertial sensors, the absolute orientation of the sensors in space.

The inertial sensors alone can determine the orientation in space only up to one rotation about the gravitational acceleration vector. In the example illustrated, the angles δ_{g}, δ₁ are shown, which are enclosed by the Earth's magnetic field M and the X axis or the horizontal axis of the mask-internal sensor 18 and of the external sensor 51 or the magnetic field sensor 51b. In the illustrated case, the alignment of the head, or of the mask 10 that is connected thereto, relative to the torso can be ascertained.

FIG. 7 shows the device according to the invention as a measurement system which additionally comprises a portable operating unit 20, a data transmission unit 30 and an evaluation unit 40. The mask 10, the apparatus 50 for capturing the body posture, and the monitoring unit 60 have already been described in detail above with reference to FIG. 1.

The operating unit 20 is connected, by way of an electric connection 21 in the form of a cable, to the measurement spectacles, i.e. to the mask 10. The connection 21 can also be wireless in the form of a radio link. The operating unit 20 comprises the power supply for the mask 10 and operating elements 22 and 23 with which the image 7 or light pattern provided on the display 12 inside the mask can be rotated clockwise or counter-clockwise. The operating unit to this end transmits control signals to the mask 10 via the electric connection 21, which effect the rotation of the image that is presented on the display 12 and is, for example, a series of dots or a line.

A further operating element 24 of the operating unit 20 serves for confirming that, after the rotation, the image 7 on the display 12 is perceived as being vertically aligned. In this case, the actuation of the operating element 24 triggers a confirmation signal, which is transmitted to the evaluation unit 40.

Measurement and/or control data are transmitted between the operating unit 20 and the evaluation unit 40 via a bidirectional radio link 35, which forms a wireless electric connection between the operating unit 20 and the data transmission unit 3a To this end, the operating unit 20 likewise comprises a data transmission unit 20a. The data transmission unit 30 is connected, via a USB connection or a connection of a similar type, to the evaluation unit 40, which is, for example, in the form of a computer unit or a PC having corresponding evaluation software.

FIGS. 8a and 8b schematically show the beam path in the mask 10 of the device 100 according to the invention, in a view from above (FIG. 8a) and from the side (FIG. 8b). Starting from the display 12 with the diffuser element 13 that is arranged before it, the image is guided via the opposite mirror device having the display mirror 16 and the main mirror 15 and subsequently through the Fresnel lens 14 to the eye 2 of the observer.

The process of a measurement of the subjective visual vertical, or SVV, using the device 100 according to the invention will be explained below by way of example with reference to FIG. 9.

The subjective visual vertical, or SVV, is defined as the adjusted angle α2 of the line 7 on the display 12, i.e. the angle between the vertically perceived line 7 and the actual z direction, or the vertical direction of the display 12 or of the mask 10 that is gathered from the tilt sensor 18. If the mask 10, and thus the head, are aligned exactly vertically, then the SVV, or the angle α2, should in the normal case be zero degrees. If the head is tilted, the angle α2 between the line 7, which is subjectively adjusted by the subject, and the z axis of the mask 10 should correspondingly increase in terms of the absolute value and should, in the ideal scenario, if the head is tilted by 90 degrees, likewise reach 90 degrees.

For the measurement according to the invention, the mask 10 is placed on a person whose SW is intended to be determined. Since the mask 10 is light-proof, no more light from the outside the mask 10 enters the eyes of the person, with the result that said person has no optical orientation of any kind. The person sits in the upright position.

Shown on the display 61 of the monitoring unit 60 is the orientation of the trunk and the head. This gives the doctor the possibility of monitoring the seated position and possibly correcting it.

Subsequently, a line 7, which clearly deviates from the perpendicular, is illustrated on the display 12 in the mask 10, which line 7 is for example a light line and is visible in the mask 10 to the subject.

The person adjusts the orientation of the line 7 using the operating unit 20 such that the line 7 appears to him or her to be perpendicular, i.e. subjectively vertical for him or her. It is thus aligned in the direction of the subjectively perceived vertical gravitational force g, as shown in FIG. 9.

As soon as this subjectively perceived vertical alignment of the line 7 has been adjusted, the subject presses the confirmation button on the operating unit 20, i.e.

the operating element 24.

The measurement is advantageously repeated several times for statistical reasons. The deviations of the individual measurement of the SW and the average value thereof from the actual vertical are processed and displayed on the monitoring unit 60. Between the individual measurements, the light line 7 on the display 12 is switched off and then switched on again in a different, randomly aligned position. The individual measurement results are stored.

After the first measurement or series of measurements, a corresponding second measurement or series of measurements is carried out according to the same sequence, but with the single difference that, in accordance with the doctor's specification, the person or patient assumes a body posture that deviates from the body posture in the first measurement operation.

Once again the monitoring unit serves here as a display which provides information relating to the instantaneous position of the head and of the remaining body regions to the doctor, who can then either confirm or correct it.

After the second measurement or series of measurements is complete, the doctor can compare, for diagnostic purposes, the values of the SVV information of both measurements, or series of measurements, that are stored in the monitoring unit 60 with one another, and, on this basis, make corresponding statements relating to a possible disorder.

For example, in the first measurement or series of measurements, the person can initially be upright or stand straight, as shown in FIG. 3a, and in the subsequent, second measurement or series of measurements, assume a second position, as is shown in FIGS. 3b and 5a. In the second position shown here, the head posture is perpendicular, while the upper body is leaning forward. Here, the person leans the upper body forward, for example, by approximately 30 degrees and lifts the head by the same angle again so as to keep the head—in absolute terms—upright again.

The invention offers new possibilities of detecting illnesses that affect the sense of balance and the otolith function. For example, illnesses in the region of the cervical spine can be detected, in which receptors in the region of the neck provide incorrect signals to the equilibrium organ.

Moreover, in the typical function test of the otoliths, it has hitherto not been possible to rule out further factors influencing the measurement, if the standard measurement position is not assumed. Such factors can be detected by way of the invention, as a result of which errors in the examination of the otolith function are reduced. That means that the informative value and the accuracy of the SVV measurement are increased, and systematic deviations owing to the measurement position are detected.

1. A device for measuring equilibrioception in persons, comprising:
an opaque mask having a tilt sensor and a display, arranged inside the mask, for presenting an image that can be rotated in order to determine a subjective visual vertical of a person; and
an apparatus for capturing body posture and/or body movement of the person, and a monitoring unit that displays and/or saves orientation and/or movement of one or more body regions during determination of the subjective visual vertical.

2. The device as claimed in claim 1, wherein the apparatus for capturing the body posture comprises one or more sensors that measure alignment and/or movement of the body or of a body part.

3. The device as claimed in claim 1, wherein the apparatus for capturing the body posture comprises one or more cameras that capture alignment and/or movement of the body, of a marker provided on the body, or of a body part.

4. The device as claimed in claim 2, wherein the at least one sensor is in a form of an inertial sensor, acceleration sensor, or a combination of acceleration sensor and angular rate sensor.

5. The device as claimed in claim 4, wherein at least one sensor and/or the tilt sensor comprise(s) a single-axis or multi-axis magnetic field sensor.

6. The device as claimed in claim 1, wherein the body posture captured by the apparatus, or a characteristic parameter thereof, is graphically displayed, wherein the display is shown on the display inside the mask and/or on a further display outside the mask.

7. The device as claimed in claim 5, wherein the monitoring unit determines, from the signals of the magnetic field sensors, angles (δₜ, δ_{g}) of one or more body regions with respect to a magnetic field and ascertains therefrom the orientation of the body regions relative to one another.

8. The device as claimed in claim 1,
comprising one or more of the following features:
(a) the opaque mask is able to be placed on a person such that it is light-proof;
(b) the signals of the tilt sensor represent tilt of the mask and/or an acceleration that acts on the mask;
(c) the vertically or horizontally aligned image orientation on the display is visually adjustable during operation by control signals, wherein a portable operating unit generates the control signals;
(d) an evaluation unit is provided that compares, in case of an image orientation that appears to be aligned vertically, horizontally, or at a different predefined angle, the tilt angle of the image relative to the mask to the signal of the tilt sensor;
(e) the tilt sensor is integrated in the mask and is in a form of a three-dimensional acceleration or inertial sensor;
(f) the image is in a form of a light pattern, a light line, an image sequence, or a video;
(g) an image memory is integrated in the mask.

9. A method for measuring equilibrioception of persons, in which an image is provided inside an opaque mask to a person, with an orientation of the image being adjusted such that it appears to the person to be aligned vertically, horizontally or at a different, previously determined angle in space, wherein a tilt sensor is used to capture a tilt of the head and/or an acceleration acting on the head in order to determine a subjective visual vertical of the person,
wherein a body posture of the person is captured and the orientation and/or movement of one or more body regions of the person is displayed and/or saved by a monitoring unit.

10. The method as claimed in claim 9, wherein the orientation of the subjective visual vertical is ascertained in dependence on the body posture.

11. The method as claimed in claim 9, wherein determination of the subjective visual vertical takes place during a defined body posture which is displayed or checked using the monitoring unit.

12. The method as claimed in claim 9, wherein in case of a first body posture and in case of a second body posture, the respective orientation of the subjective visual vertical is measured, and both results are compared to one another.

13. The method as claimed in claim to 12, wherein, during measurement, the orientation of individual body regions relative to one another is determined, wherein in particular relative orientation between head and trunk is determined.

14. The method as claimed in claim 9, characterized in that wherein the captured body posture, or a characteristic parameter thereof, is graphically displayed, wherein the display is shown on the display inside the mask and/or on a further display outside the mask.

15. The method as claimed in claim 9, wherein the image is presented on an electronic display inside the mask, and the image is rotated, using control signals, about an observation direction until it appears to the person to be aligned vertically, horizontally, or at another angle previously to be determined in space.

16. The method as claimed in claim 9, wherein the person adjusts the subjectively perceived vertical image orientation him or herself using a portable operating unit.

17. The method as claimed in claim 9, wherein a device for measuring equilibrioception in persons as is used, the device comprising:
an opaque mask having a tilt sensor and a display, arranged inside the mask, for presenting an image that can be rotated in order to determine a subjective visual vertical of a person; and
an apparatus for capturing body posture and/or body movement of the person, and a monitoring unit that displays and/or saves orientation and/or movement of one or more body regions during determination of the subjective visual vertical.

18. The method as claimed in claim 17, comprising use for measuring effect of the body posture on equilibrioception.
